(19) United States
(12) Patent Application Publication
(10) Pub. No.: US 2020/0093460 A1
(43) Pub. Date: Mar. 26, 2020

(54) METHOD, DEVICE, ULTRASONIC PROBE AND TERMINAL FOR ADJUSTING DETECTION POSITION

(71) Applicant: CLOUDMINDS (SHENZHEN) HOLDINGS CO., LTD., Shenzhen (CN)

(72) Inventor: Lei LUO, Shenzhen (CN)

(21) Appl. No.: 16/579,929

(22) Filed: Sep. 24, 2019

(30) Foreign Application Priority Data
Sep. 26, 2018 (CN) 201811125772.2

(51) Int. Cl. A61B 8/08; G06T 7/73; G06T 7/00; A61B 8/00

(52) U.S. Cl. CPC A61B 8/0833; G06T 7/74; G06T 7/97; G06T 2207/10132; A61B 8/4245; A61B 8/46; A61B 8/5207; G06T 7/75

(57) ABSTRACT

Embodiments of the present disclosure relate to the field of ultrasonic detection, and disclose a method, device, ultrasonic probe, and terminal for adjusting a detection position. The method for adjusting a detection position in the present disclosure comprises: acquiring a first ultrasonic image currently detected by a ultrasonic probe; determining a current detection position of the detected object corresponding to the first ultrasonic image; determining indication information for indicating movement of the ultrasonic probe, according to the current detection position of the detected object and a target detection position of the detected object; and adjusting the detection position of the ultrasonic probe according to the indication information. In the present disclosure, the user may quickly acquire the correct ultrasonic detection image.

### CROSS-REFERENCE TO RELATED APPLICATION

This application claims the priority benefit of Chinese Patent Application No. 201811125772.2 filed on Sep. 26, 2018 and entitled “METHOD, DEVICE AND ULTRASONIC PROBE AND TERMINAL FOR ADJUSTING DETECTION POSITION”, the disclosure of which is incorporated by reference herein in its entirety.

### TECHNICAL FIELD

Embodiments of the present disclosure relate to the field of ultrasonic detection, and in particular, to a method, device, ultrasonic probe and terminal for adjusting a detection position.

### BACKGROUND

With the development of technology, miniaturized ultrasonic detection apparatus, such as handheld ultrasonic detection apparatus has emerged. Small ultrasonic detection apparatus is no longer limited to be used in hospital, but may also be used in small health stations, or be carried by a doctor when visiting a patient at home, or even be purchased by users for home usage.

While studying the technologies in the related art, the inventor found that when performing ultrasonic detection, a user needs to use an ultrasonic detection probe at a correct detection position with a correct detection angle so as to acquire a correct ultrasonic detection image. However, for doctors in small health stations and ordinary users who have purchased the ultrasonic detection apparatus by themselves, it is very difficult to perform ultrasonic detection at a correct detection position with a correct detection angle when using a small ultrasonic detection apparatus.

Obviously, how to quickly acquire the correct ultrasonic detection image for a user without expertise in using the ultrasonic detection apparatus is a problem that needs to be solved.

### SUMMARY

One of objects of some embodiments of the present disclosure is to provide a method, device, ultrasonic probe, and terminal for adjusting a detection position so that a user may quickly acquire a correct ultrasonic detection image.

In order to solve the above technical problem, the embodiments of the present disclosure provide a method for adjusting a detection position, comprising: acquiring a first ultrasonic image currently detected by a ultrasonic probe; determining a current detection position of a detected object corresponding to the first ultrasonic image; determining indication information for indicating movement of the ultrasonic probe, according to the current detection position of the detected object and a target detection position of the detected object; and adjusting the detection position of the ultrasonic probe according to the indication information.

Embodiments of the present disclosure further provide a device for adjusting a detection position, comprising: an acquisition module, a first determination module, a second determination module, and an indication module; the acquisition module is configured to acquire a first ultrasonic image currently detected by a ultrasonic probe; the first determination module is configured to determine a current detection position of a detected object corresponding to the first ultrasonic image; the second determination module is configured to determine, according to the current detection position of the detected object and a target detection position of the detected object, indication information for indicating movement of the ultrasonic probe; and the indication module is configured to adjust a detection position of the ultrasonic probe according to the indication information.

Embodiments of the present disclosure further provide an ultrasonic probe, comprising: a communication module, an ultrasonic detection module, and an indication module; the ultrasonic detection module is configured to acquire an ultrasonic signal of a current detection position; the communication module is configured to send the ultrasonic signal, and receive indication information for indicating movement of the ultrasonic probe; and the indication module is configured to output the indication information.

Embodiments of the present disclosure further provide a terminal, comprising: at least one processor; and a memory communicatively coupled to the at least one processor; wherein the memory stores instructions executable by the at least one processor, the instructions being executed by the at least one processor to enable the at least one processor to perform the method for adjusting a detection position.

Compared with the prior art, in the embodiments of the present disclosure, the ultrasonic image for the target detection position of the detected object is the most accurate one among ultrasonic images of the detected object, and the currently detected first ultrasonic image is acquired in real time, so that the current detection position of the detected object corresponding to the first ultrasonic image is determined in real time; the accurate indication information is determined in real time according to the current detection position of the detected object and the target detection position of the detected object, which ensures the accuracy of adjustment of the ultrasonic probe performed each time, and increases the speed at which the ultrasonic probe is adjusted to the target detection position of the detected object; the detection position of the ultrasonic probe is adjusted according to the indication information, such that the user may accurately acquire the correct ultrasonic image of the detected object even without professional knowledge about ultrasonic detection, thus improving the applicability of the ultrasonic detection apparatus.

In addition, the determining a current detection position of a detected object corresponding to the first ultrasonic image specifically comprises: determining a detected object corresponding to the first ultrasonic image; and determining position information corresponding to an area of the detected object that matches the first ultrasonic image, and taking a position indicated by the position information as the current detection position of the detected object corresponding to the first ultrasonic image. The detected object is firstly determined, and then only the area of the detected object that matches the first ultrasonic image is determined in order to determine the current detection position of the detected object; thus the range for determining the current detection position is reduced, thereby the speed at which the current detection position of the detected object is determined is increased.

In addition, determining a current detection position of the detected object corresponding to the first ultrasonic image specifically comprises: acquiring a first correspondence relationship between ultrasonic images and detection positions of the detected object; and determining the current detection position of the detected object corresponding to the first ultrasonic image, according to the first ultrasonic image and the first correspondence relationship. The current detection position of the detected object corresponding to the first ultrasonic image can be determined by the first ultrasonic image and the first correspondence relationship, thus the operation is simple and the speed is fast.

In addition, the acquiring a first correspondence relationship between the ultrasonic image and the detection position of the detected object specifically comprises: determining the detected object corresponding to the first ultrasonic image; and acquiring, according to the detected object, the first correspondence relationship between ultrasonic images and detection positions of the detected object. Since the detected object is firstly determined, the range for looking up the first correspondence relationship is reduced, thereby the speed for acquiring the first correspondence relationship is increased.

In addition, the determining the detected object corresponding to the first ultrasonic image specifically comprises: determining the detected object corresponding to the first ultrasonic image according to the first ultrasonic image and a second correspondence relationship, wherein the second correspondence relationship is a correspondence relationship between the ultrasonic images and the detected object, and the second correspondence relationship is determined in advance according to at least two ultrasonic images corresponding to each detected object. By determining the detected object according to the second correspondence relationship, and the operation is simplified and the speed is improved.

In addition, determining the detected object corresponding to the first ultrasonic image specifically comprises: matching the first ultrasonic image with pre-stored first three-dimensional models for each object to be detected to determine a first three-dimensional model that is successfully matched, and taking an object to be detected corresponding to the first three-dimensional model that is successfully matched as the detected object corresponding to the ultrasonic image. By performing matching with the pre-stored first three-dimensional model of the object to be detected, the detected object corresponding to the first ultrasonic image can be accurately determined.

In addition, the determining indication information for indicating movement of the ultrasonic probe, according to the current detection position of the detected object and a target detection position of the detected object specifically comprises: calculating information on a relative position between the current detection position and the target detection position of the detected object; and taking the information on the relative position as the indication information. The information on the relative position is directly calculated and thus the calculation is simple.

In addition, the adjusting the detection position of the ultrasonic probe according to the indication information specifically comprises: sending the indication information to the ultrasonic probe, outputting the indication information by the ultrasonic probe, and adjusting, by the user, the detection position of the ultrasonic probe according to the indication information; or, outputting the indication information, and adjusting, by the user, the detection position of the ultrasonic probe according to the indication information. The user adjusts the detection position of the ultrasonic probe according to the output indication information, and the indication information may be output by the ultrasonic probe or may be directly output, such that the manner of outputting the indication information is more flexible.

In addition, the method for adjusting a detection position further comprises performing the following step after determining the indication information for indicating movement of the ultrasonic probe: if the indication information is determined as indicating that the current detection position of the detected object is the same as the target detection position of the detected object, saving the first ultrasonic image. According to the method for adjusting a detection position, only the first ultrasonic image belonging to the target detection position of the detected object is saved, that is, only the correct ultrasonic image is saved, so the storage space is saved, and at the same time, the accuracy of subsequent analysis of the detected object is ensured.

In addition, the method for adjusting a detection position further comprises performing the following step after saving the first ultrasonic image: determining whether there is a next target detection position for the detected object; and if yes, determining indication information for indicating the movement of the ultrasonic probe to the next target detection position according to the current detection position of the detected object; or if not, outputting the saved ultrasonic image corresponding to each target detection position.

In addition, the indication information comprises: a direction for the ultrasonic probe to adjust and an angle for the ultrasonic probe to rotate. The indication information comprising a direction for the ultrasonic probe to adjust and an angle for the ultrasonic probe to rotate ensures that the ultrasonic probe is at a correct angle when detecting the target detection position of the detected object to be detected, ensuring that the acquired ultrasonic image is correct.

### BRIEF DESCRIPTION OF THE DRAWINGS

One or more embodiments are exemplarily described by using figures in the accompanying drawings corresponding thereto. The exemplary descriptions do not constitute a limitation on the embodiments. Elements with a same reference numeral in the accompanying drawings represent similar elements. Unless otherwise particularly stated, the figures in the accompanying drawings do not constitute a limitation.

FIG. 1 is a specific flowchart of a method for adjusting a detection position according to an embodiment of the present application;

FIG. 2 is a specific flowchart of determining a current detection position of a detected object corresponding to a first ultrasonic image in a method for adjusting a detection position according to another embodiment of the present application;

FIG. 3 is a specific flowchart of a method for adjusting a detection position according to another embodiment of the present application;

FIG. 4 is a specific structural diagram of a device for adjusting a detection position according to another embodiment of the present application;

FIG. 5 is a specific structural diagram of an ultrasonic probe according to another embodiment of the present application;

FIG. 6 is a specific structural diagram of an indication module in an ultrasonic probe according to another embodiment of the present application;

FIG. 7 is a diagram showing a specific arrangement of an indicator light in an ultrasonic probe according to another embodiment of the present application;

FIG. 8 is a structural diagram of a terminal according to another embodiment of the present application;

FIG. 9 is a structural diagram of an ultrasonic detection apparatus according to another embodiment of the present application.

### DETAILED DESCRIPTION

To make objects, technical solutions, advantages of the embodiments of the present disclosure clearer, the embodiments of the present disclosure will be described in detail below with reference to the accompanying drawings. However, it may be appreciated by those skilled in the art that, in various embodiments of the present disclosure, numerous technical details are set forth in order to provide the reader with a better understanding of the present application. However, the technical solutions claimed in the present application can be implemented without these technical details and various changes and modifications based on the following embodiments.

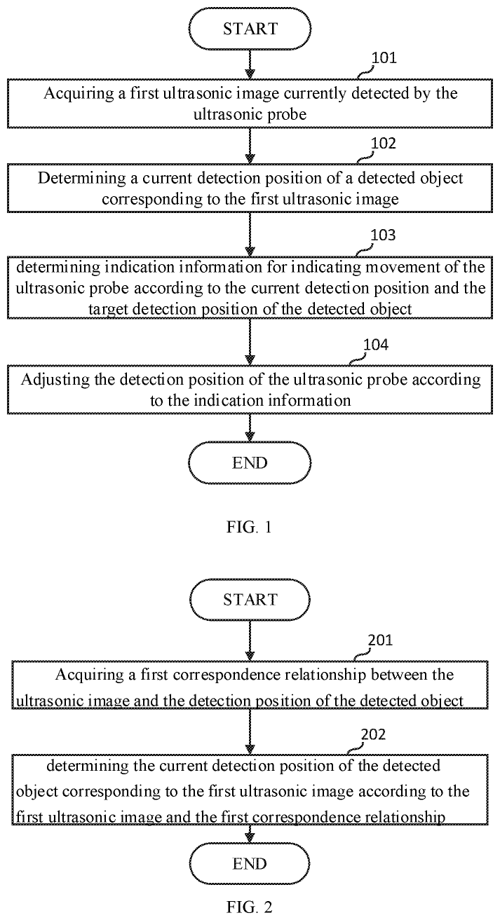

An embodiment of the present disclosure relates to a method for adjusting a detection position, which may be applied to an ultrasonic detection apparatus, for example, a hand-held ultrasonic detection apparatus. Generally, the ultrasonic detection apparatus comprises an ultrasonic probe and a processing end communicatively coupled with the ultrasonic probe, and the processing end is configured to receive an ultrasonic signal acquired by the ultrasonic probe. In the present embodiment, an example is taken in which the method for adjusting a detection position is applied to an processing end in an ultrasonic detection apparatus. The specific flow of the method for adjusting a detection position is shown in FIG. 1.

In the embodiment, the specific process for adjusting a detection position is described by taking human body detection as an example:

Step 101: acquiring a first ultrasonic image currently detected by an ultrasonic probe.

Specifically, the processing end may be a apparatus having a display function, such as a smart phone or a computer; and the processing end may also be a remote server or the like; this embodiment does not limit the type of the processing end. The processing end is communicatively coupled to the ultrasonic probe via wireless or wired connection. The ultrasonic probe sends a currently detected ultrasonic signal back to the processing end, and the processing end generates a first ultrasonic image according to the ultrasonic signal.

The user may place the ultrasonic probe on a surface of a human body to detect an organ to be detected. It will be understood that in order to speed up the detection of the organ to be detected, the user may place the probe on a position of the surface of the human body that substantially corresponds to the organ to be detected, and the position at which the ultrasonic probe is initially placed does not have to be precise.

Step 102: determining a current detection position of a detected object corresponding to the first ultrasonic image.

Specifically, a first three-dimensional model for each of objects to be detected is pre-stored. Of course, a relative position relationship among the first three-dimensional models for respective objects to be detected may be pre-stored while pre-storing the first three-dimensional model for each of objects to be detected. In this embodiment, each organ of the human body may be taken as an object to be detected. For example, a first three-dimensional model for each of organs in the human body is constructed, and a three-dimensional model for all human organs is constructed according to the relative position relationship among respective organs in the human body.

It will be understood that the pre-stored first three-dimensional model for each of the objects to be detected may include coordinate information and angle information of each of detection points of the object to be detected, for example, each of the pre-stored organs to be detected of the human body, and coordinate information and angle information of medical ultrasonic detection points of each of the organ to be detected.

In a specific implementation, the specific process for determining a current detection position of the detected object corresponding to the first ultrasonic image may comprises: determining a detected object corresponding to the first ultrasonic image; and determining position information corresponding to an area of the detected object that matches the first ultrasonic image, and taking a position indicated by the position information as the current detection position of the detected object corresponding to the first ultrasonic image.

The determination on the detected object corresponding to the first ultrasonic image may be implemented in the following two manners:

Manner 1: determining the detected object corresponding to the first ultrasonic image, according to the first ultrasonic image and a second correspondence relationship between ultrasonic images and the detected object, and it is determined in advance according to at least two ultrasonic images corresponding to the detected object.

Specifically, the second correspondence relationship between the ultrasonic images and each of objects to be detected may be constructed in advance according to at least two ultrasonic images corresponding to each of the objects to be detected. In this embodiment, each organ of the human body may be taken as an object to be detected, and a large number of ultrasonic images corresponding to each organ may be acquired. Through performing a deep learning on the ultrasonic images of each of the objects to be detected, the second correspondence relationship between the ultrasonic images and the each of the objects to be detected may be constructed. For example, 100 ultrasonic images corresponding to the human heart, 100 ultrasonic images corresponding to the human stomach, and 100 ultrasonic images corresponding to the human lung are acquired, and the 100 ultrasonic images of each organ may include ultrasonic images of the organ taken at different angles. The second correspondence relationship between the ultrasonic images and each of the objects to be detected may be constructed in advance by a deep learning algorithm. At this time, by using the second correspondence relationship, only an ultrasonic image needs to be input, and the detected object corresponding to the ultrasonic image may be determined by identifying features in the ultrasonic image. The specific way of deep learning is not described here.

Manner 2: performing matching on the first ultrasonic image and the pre-stored first three-dimensional model of each of the objects to be detected, determining a first three-dimensional model that is successfully matched, and taking the object to be detected corresponding to the first three-dimensional model that is successfully matched as the detected object corresponding to the ultrasonic image.

Specifically, a matching between the first ultrasonic image and the pre-stored first three-dimensional model of each of the objects to be detected is performed, the similarity between the first ultrasonic image and the pre-stored first three-dimensional model of each of the objects to be detected is acquired, and if the similarity exceeds a preset threshold, the matching is considered to be successful, and the object to be detected corresponding to the first three-dimensional model that is successfully matched is taken as the detected object corresponding to the first ultrasonic image.

For example, a matching of an ultrasonic image A with a pre-stored first three-dimensional model for each of three objects to be detected (that is, heart, stomach and lung) is performed respectively, the relative position relationship among the heart, the stomach and the lung is pre-stored, and the preset threshold for similarly is set as 90%. If a similarity between the ultrasonic image A and the first three-dimensional model for the heart is 95%, a similarity between the ultrasonic image A and the first three-dimensional model for the stomach is 20%, and a similarity between the ultrasonic image A and the first three-dimensional model for the lung is 10%, the ultrasonic image A is successfully matched with the first three-dimensional model for the heart, and the detected object corresponding to the ultrasonic image A is determined as the heart.

After determining the detected object corresponding to the first ultrasonic image, position information corresponding to an area in the detected object that matches the first ultrasonic image is acquired, and the position indicated by the position information is taken as the current detection position of the detected object corresponding to the first ultrasonic image.

It is worth mentioning that if the current detection position of the detected object corresponding to the first ultrasonic image cannot be determined, it indicates that the ultrasonic probe is currently out of a detection range, and an error prompt may be directly output.

Step 103: determining indication information for indicating movement of the ultrasonic probe according to the current detection position of the detected object and a target detection position of the detected object.

In a specific implementation, information on a relative position between the current detection position and the target detection position of the detected object is calculated; and the information on a relative position is taken as the indication information. The indication information comprises a direction for the ultrasonic probe to adjust and an angle for the ultrasonic probe to rotate.

Specifically, the target detection position of each of the objects to be detected is pre-stored, and after the detected object is determined, the target detection position of the detected object may be acquired. At this time, a coordinate system may be constructed for the detected object. The relative position relationship between the current detection position and the target detection position of the detected object is calculated by using the constructed coordinate system. For example, a difference between the target detection position of the detected object and the current detection position of the detected object is calculated, the angle difference and the direction difference therebetween is determined, and the direction difference and the angle difference are taken as the indication information. The indication information indicates the direction and the rotation angle for the ultrasonic probe to move to the target detection position, for example, the indication information may include moving to the left and rotating clockwise.

Step 104: adjusting the detection position of the ultrasonic probe according to the indication information.

In a specific implementation, the indication information is sent to and thus output by the ultrasonic probe, and the user may adjust the detection position of the ultrasonic probe according to the indication information; or the indication information is output, and the user may adjust the detection position of the ultrasonic probe according to the indication information.

Specifically, the indication information is sent to the ultrasonic probe, the ultrasonic probe may output the indication information by means of an indicator light, or the indication information may also be output by playing a prompt tone. It will be understood that the processing end may also directly output the indication information and the way for the output is not limited to: displaying a prompt message or playing a prompt tone. The user may adjust the detection position of the ultrasonic probe according to the output indication information.

It should be noted that after each detection of the ultrasonic probe, steps 101 to 104 may be repeated for continuously instructing the ultrasonic probe to adjust the detection position. Of course, if the current detection position and the target detection position of the detected object are the same, the determined indication information may include a prompt message of “Adjustment Ended”; or the indication information is empty. The implementation of the prompt message will not be limited here, and it may be set according to actual needs.

Compared with the prior art, in the embodiments of the present disclosure, the ultrasonic image for the target detection position of the detected object is the most accurate one among ultrasonic images of the detected object, and the currently detected first ultrasonic image is acquired in real time, so that the current detection position of the detected object corresponding to the first ultrasonic image is determined in real time; the accurate indication information is determined in real time according to the current detection position of the detected object and the target detection position of the detected object, which ensures the accuracy of adjustment of the ultrasonic probe performed each time, and increases the speed at which the ultrasonic probe is adjusted to the target detection position of the detected object; the detection position of the ultrasonic probe is adjusted according to the indication information, such that the user may accurately acquire the correct ultrasonic image of the detected object even without professional knowledge about ultrasonic detection, thus improving the applicability of the ultrasonic detection apparatus.

Another embodiment of the present disclosure relates to a method for adjusting a detection position. This embodiment is substantially the same as the embodiment described with reference to FIG. 1, and the main difference is that, in this embodiment of the present disclosure, the current detection position of the detected object corresponding to the first ultrasonic image is determined by acquiring a first correspondence relationship between ultrasonic images and detection positions of the detected object. In the following, the process for determining the current detection position of the detected object corresponding to the first ultrasonic image in the embodiment is specifically described. The specific process of this step is shown in FIG. 2.

Step 201: acquiring a first correspondence relationship between ultrasonic images and detection positions of the detected object.

Specifically, the first correspondence relationship between ultrasonic images and detection positions of the detected object is constructed in advance, and there are two manners for constructing the first correspondence relationship, which will be specifically described below.

Manner 1:

A large amount of construction data is acquired in advance, and the construction data may be acquired from a cloud or may be input in advance by an engineer, wherein the construction data comprises ultrasonic images of each detected object, position information of each detected object, and a target detection position of each detected object. The first correspondence relationship between the ultrasonic images and detection positions of the detected object is determined by means of deep learning. After the first correspondence relationship is constructed, only when the first ultrasonic image needs to be input, the detected object corresponding to the first ultrasonic image and the current detection position of the detected object corresponding to the first ultrasonic image may be acquired according to the first correspondence relationship.

Manner 2:

Similar to the first manner, a large amount of construction data is acquired in advance, and the construction data comprises a ultrasonic image of each detected object, position information of each detected object, and a target detection position of each detected object. A second correspondence relationship between ultrasonic images and the detected objects are constructed in advance by employing a deep learning algorithm according to a large amount of construction data; and a correspondence relationship between ultrasonic images of the detected object and detection positions of the detected object is constructed for each detected object. That is, the first correspondence relationship between ultrasonic images and detection positions of the detected object may be determined according to the detected object.

If the first correspondence relationship is constructed in advance by using manner 1, the first correspondence relationship may be directly acquired. If the first correspondence relationship is constructed by using manner 2, the process for acquiring the first correspondence relationship comprises: determining the detected object corresponding to the first ultrasonic image; acquiring the first correspondence relationship between the ultrasonic image and the detection position of the detected object according to the detected object.

It will be understood that, determining the detected object corresponding to the first ultrasonic image in this embodiment may be implemented in the two manners provided in the embodiment described with reference to FIG. 1, and the two manners for determining the detected object are not described here. However, it should be noted that if the detected object is determined by using manner 2 in the embodiment described with reference to FIG. 1, the first three-dimensional model corresponding to each object to be detected needs to be pre-stored.

Step 202: determining the current detection position of the detected object corresponding to the first ultrasonic image according to the first ultrasonic image and the first correspondence relationship.

Specifically, the detection position of the detected object corresponding to the first ultrasonic image may be determined according to the first correspondence relationship and the first ultrasonic image.

The method for adjusting a detection position provided by the embodiment may determine the current detection position of the detected object corresponding to the first ultrasonic image according to the first ultrasonic image and the first correspondence relationship, and the operation is simple and the speed is fast. At the same time, a plurality of manners for constructing the first correspondence relationship as well as acquiring the first correspondence relationship are provided, thus improving the flexibility of determining the current detection position of the detected object corresponding to the first ultrasonic image.

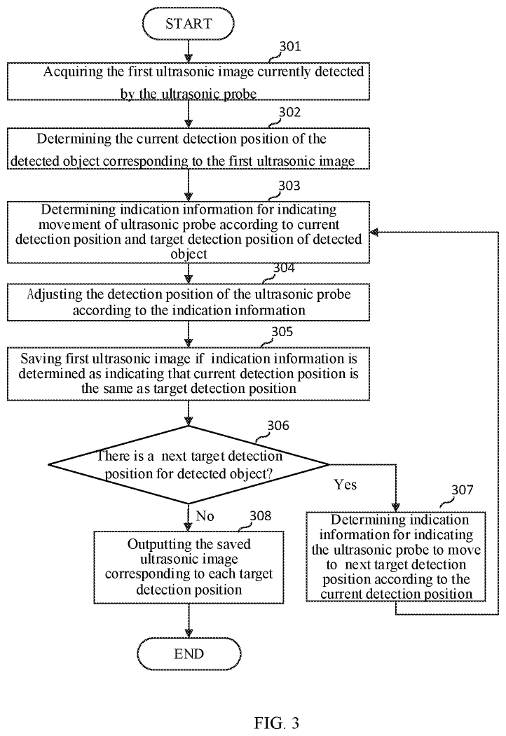

Another embodiment of the present disclosure relates to a method for adjusting a detection position, and this embodiment provides a further improvement based on the embodiments described with reference to FIG. 1 or FIG. 2, the main improvement comprises: after the indication information for indicating movement of the ultrasonic probe is determined, saving the first ultrasonic image, if the indication information is determined as indicating that the current detection position of the detected object is the same as the target detection position of the detected object. The specific flow diagram is shown in FIG. 3.

Step 301: acquiring a first ultrasonic image currently detected by a ultrasonic probe.

Step 302: determining a current detection position of a detected object corresponding to the first ultrasonic image.

Step 303: determining indication information for indicating movement of the ultrasonic probe according to the current detection position of the detected object and a target detection position of the detected object.

Step 304: adjusting a detection position of the ultrasonic probe according to the indication information.

Step 305: saving the first ultrasonic image, if the indication information is determined as indicating that the current detection position of the detected object is the same as the target detection position of the detected object.

Specifically, if the indication information is determined as indicating that the current detection position of the detected object is the same as the target detection position of the detected object, it indicates that the current detection position of the detected object and the target detection position of the detected object coincide, and at this time, the first ultrasonic image is saved.

Step 306: judging whether there is a next target detection position for the detected object, and if yes, step 307 is performed, if not, step 308 is performed.

Specifically, a plurality of target detection positions may be set for the detected object, and the plurality of target detection positions of the detected object may be sorted in advance, and the ultrasonic probe is sequentially adjusted to reach respective target detection positions according to a preset order, wherein the order of the target detection positions is not limited and may be set according to actual needs. For example, three target detection positions are preset for a human heart, namely position A, position B, and position C, and the three target detection positions are sorted in advance, the order of the sorted target detection points may be position C, position B, and position A. If it is determined that the current detection position coincides with the target detection position C, it may be learned that there is still a next target detection position B according to the order; and if it is determined that the current detection position coincides with the target detection position A, it may be learned according to the order that a next target detection position does not exist.

Step 307: determining indication information for the ultrasonic probe to move to the next target detection position according to the current detection position of the detected object.

Specifically, the manner in step 303 may be adopted, the ultrasonic probe is adjusted to the next target detection position according to the determined indication information, based on the current detection position of the detected object and the indication information of the next target detection position, and the ultrasonic image of the next target detection position is saved.

Step 308: outputting the saved ultrasonic image corresponding to each of target detection positions.

Specifically, if there are a plurality of target detection positions for the detected object, the ultrasonic image corresponding to each of the target detection positions is output, and the outputting may be implemented by sending the ultrasonic image corresponding to each of the target detection positions to the user's terminal or directly displaying the same, such that the user may analyze, study the ultrasonic image of each of the target detection positions.

It should be noted that steps 301 to 304 in the present embodiment is substantially the same as steps 101 to 104 in the embodiment described with reference to FIG. 1, and thus will not be repeated here. Step 305 in the embodiment may be performed after step 303, that is, performing step 303, step 305, step 304, and step 306 sequentially.

The method for adjusting a detection position provided in the embodiment only saves the first ultrasonic image for the target detection position of the detected object, that is, only the correct ultrasonic image is saved, thereby saving storage space while ensuring the accuracy for analyzing the ultrasonic image of the detected object in the subsequent process; the saved ultrasonic image of each target detection position is output, such that the user may analyze the correct ultrasonic image of each target detection position and acquire the correct analysis result.

Division of steps of the foregoing methods is made for the purpose of clear description, and during implementation, the steps may be combined into one step or some steps may be split into a plurality of steps. Provided that a same logical relationship is included, the division falls within the protection scope of this patent application. Unnecessary modifications or unnecessary designs added/introduced to an algorithm or a procedure also fall within the protection scope of this patent application as long as a core design of the algorithm or the procedure is not change.

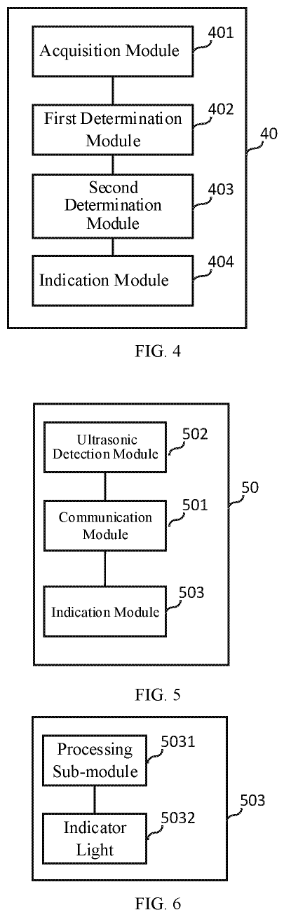

Another embodiment of the present disclosure relates to a device for adjusting a detection position. The device for adjusting a detection position 40 comprises an acquisition module 401, a first determination module 402, a second determination module 403, and an indication module 404. The specific structure is shown in FIG. 4.

The acquisition module 401 is configured to acquire a first ultrasonic image currently detected by the ultrasonic probe; the first determining module 402 is configured to determine a current detection position of a detected object corresponding to the first ultrasonic image; the second determining module 403 is configured to determine the indication information for indicating movement of the ultrasonic probe according to the current detection position of the detected object and the target detection position of the detected object; and the indication module 404 is configured to adjust the detection position of the ultrasonic probe according to the indication information.

It is not difficult to find that, this embodiment is a system embodiment corresponding to the embodiment described with reference to FIG. 1, and thus it may be implemented in cooperation with the embodiment described with reference to FIG. 1. Related technical details mentioned in the embodiment described with reference to FIG. 1 are still valid in this embodiment, and details are not described herein again in order to avoid repetition. Accordingly, the related technical details mentioned in this embodiment may also be applied to the embodiment described with reference to FIG. 1.

It should be noted that, the various modules in this embodiment are logical modules, and in an actual application, a logical unit may be a physical unit, or may be a part of a physical unit, or may be implemented by a combination of a plurality of physical units. In addition, to highlight a creative part of the present disclosure, units not closely related to the technical problem proposed in the present disclosure are not introduced in this embodiment. However, it does not indicate that there are no other units in this embodiment.

Another embodiment of the invention relates to an ultrasonic probe. The ultrasonic probe 50 comprises a communication module 501, an ultrasonic detection module 502, and an indication module 503; and the specific structure is shown in FIG. 5.

The ultrasonic detection module 502 is configured to acquire an ultrasonic signal of a current detection position; the communication module 501 is configured to send the ultrasonic signal, and receive indication information for indicating movement of the ultrasonic probe; and the indicating module 503 is configured to output the indication information.

Specifically, the ultrasonic detection module 502 acquires an ultrasonic signal of the current detection position, for example, the ultrasonic probe 50 is placed on a surface of a human skin, the ultrasonic detection module 502 acquires the ultrasonic signal of the current detection position, and transmits the ultrasonic signal to the communication module. 501, the ultrasonic signal is sent by the communication module 501 to a processing end of an ultrasonic detection device, and the processing end acquires the first ultrasonic image of the current detection position according to the ultrasonic signal, and determines the current detection position of the detected object corresponding to the first ultrasonic image, and determines indication information for indicating movement of the ultrasonic probe 50 according to the current detection position of the detected object and the target detection position of the detected object; the ultrasonic probe 50 receives the indication information returned by the processing end through the communication module 501, and transmits the indication information to the indication module 503, and the indication information is output by the indication module 503. The output manner of the indication module 503 may be means of lighting the corresponding indicator light or emitting a prompt sound, which is not limited in this embodiment.

The ultrasonic probe provided by the embodiment has an indication module, and may output the acquired indication information, such that the user may adjust the detection position of the probe according to the output indication information, and the user may be assisted to quickly adjust the ultrasonic probe to the correct detection position of the detected object.

Another embodiment of the present disclosure relates to an ultrasonic probe, and this embodiment provides a further refinement based on the instruction module 503 in the embodiment described with reference to FIG. 5. The indication module 503 comprises: a processing sub-module 5031 and an indicator light 5032, and the specific structure thereof is shown in FIG. 6.

Specifically, the processing sub-module 5031 is connected to the indicator light 5032, and the processing sub-module 5031 is configured to acquire instruction information from the communication module 501, and control the indicator light 5032 to be turned on or turned off according to the indication information. The indication information comprises a direction for the ultrasonic probe to adjust 50 and an angle for the ultrasonic probe to rotate 50. The indicator light also comprises a direction indicator light and an angle indicator light. The processing sub-module 5031 adjusts the direction indicator light to be turned on or turned off according to the direction information in the indication information. Similarly, the processing sub-module 5031 adjusts the angle indicator light to be turned on or turned off according to the angle information in the indication information.

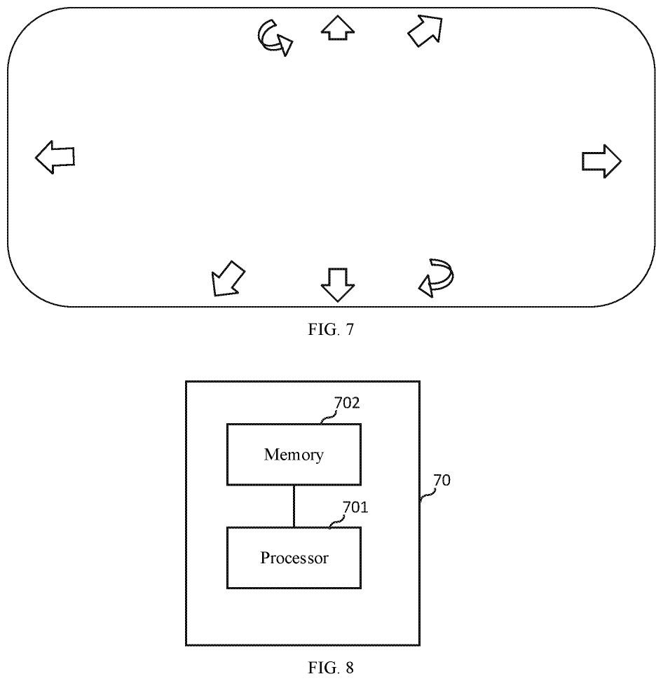

It will be understood that, as shown in FIG. 7, the direction indicator lights of four directions, i.e., up, down, left, and right can be set, and the angle indicator lights of two rotation directions can be set, that is, a left angle indicator light, and a right angle indicator light. Of course, the number and direction of the direction indicator light, and the number and rotation angle of the angle indicator light can be set according to actual needs, and are not limited to the manners listed in FIG. 7.

The ultrasonic probe provided in the embodiment adopts an indicator light to output indication information, such that the indication is clear, and the user can adjust the ultrasonic probe accordingly.

Another embodiment of the present disclosure relates to a terminal. The terminal 70 comprises: at least one processor 701; and a memory 702 communicatively coupled to at least one processor 701; wherein the memory 702 stores instructions executable by the at least one processor 701, and the instructions is executed by the at least one processor 701, such that the at least one processor 701 can implement the method for adjusting the detection position. The specific structure is shown in FIG. 8.

The memory 702 and the processor 701 are connected via a bus, the bus may include any number of mutually connected buses and bridges, and the bus connects one or more processors 701 with various circuits of the memory 702. The bus may further connect various other circuits such as a peripheral device, a voltage regulator, and a power management circuit, which are well known in the art, and therefore will not further described. The bus interface provides an interface between the bus and a transceiver. The transceiver may be one element, or may be a plurality of elements, for example, a plurality of receivers and transmitters, which provides units configured to communicate with various other apparatuses. Data processed by the processor is transmitted over a wireless media by using an antenna, and the antenna further receives the data and transfers the data to the processor.

The processor is in charge of bus managing and general processing, and may further provide various functions, including timing, a peripheral interface, voltage adjustment, power supply management, and other control functions. The memory may be configured to store data used when the processor performs an operation.

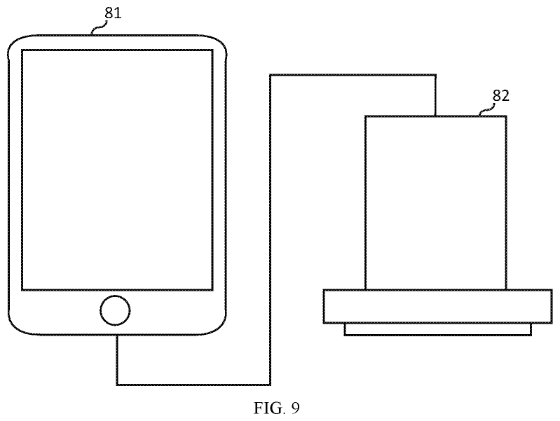

Another embodiment of the present application relates to an ultrasonic detection apparatus, as shown in FIG. 9. The ultrasonic detection apparatus includes the device 81 for adjusting a detection position according to the embodiment described with reference to FIG. 5 and the ultrasonic probe 82 according to the embodiments described with reference to FIG. 6 or FIG. 8.

Specifically, the ultrasonic detection apparatus may be an ultrasonic device applied to a human body inspection, or may be an ultrasonic device for ultrasonic detection of components. For specific applications, it is not limited here.

Those skilled in the art will understand that all or some of the steps in the foregoing method embodiments may be implemented by related hardware instructed through a program. The program is stored in one storage medium, and includes several instructions to cause a device (which may be a single-chip microcomputer, a chip, or the like) or the processor to perform all or some of the steps in the methods in the embodiments of the present disclosure. The foregoing storage medium includes various media that may store program code, for example, a USB flash drive, a removable hard disk, a read-only memory (ROM,), a random access memory (RAM), a magnetic disk, or an optical disc.

Those skilled in the art may understand that the foregoing embodiments are specific embodiments for implementing the present disclosure, and various modifications may be made to the embodiments in forms and in details during actual application without departing from the spirit and scope of the present disclosure.

What is claimed is:

1. A method for adjusting a detection position, comprising:
acquiring a first ultrasonic image currently detected by an ultrasonic probe;
determining a current detection position of a detected object corresponding to the first ultrasonic image;
determining indication information for indicating movement of the ultrasonic probe, according to the current detection position of the detected object and a target detection position of the detected object; and
adjusting the detection position of the ultrasonic probe according to the indication information.

2. The method for adjusting a detection position according to claim 1, wherein the determining a current detection position of a detected object corresponding to the first ultrasonic image specifically comprises:
determining a detected object corresponding to the first ultrasonic image; and
determining position information corresponding to an area of the detected object that matches the first ultrasonic image, and taking a position indicated by the position information as the current detection position of the detected object corresponding to the first ultrasonic image.

3. The method for adjusting a detection position according to claim 2, wherein the determining the detected object corresponding to the first ultrasonic image specifically comprises:
determining the detected object corresponding to the first ultrasonic image according to the first ultrasonic image and a second correspondence relationship between ultrasonic images and the detected object, wherein the second correspondence relationship is determined in advance according to at least two ultrasonic images corresponding to the detected object.

4. The method for adjusting a detection position according to claim 2, wherein determining the detected object corresponding to the first ultrasonic image specifically comprises: matching the first ultrasonic image with pre-stored first three-dimensional models for respective objects to be detected to determine a first three-dimensional model that is successfully matched, and taking an object to be detected corresponding to the first three-dimensional model that is successfully matched as the detected object corresponding to the ultrasonic image.

5. The method for adjusting a detection position according to claim 1, wherein the determining a current detection position of the detected object corresponding to the first ultrasonic image specifically comprises:
acquiring a first correspondence relationship between ultrasonic images and detection positions of the detected object; and
determining the current detection position of the detected object corresponding to the first ultrasonic image, according to the first ultrasonic image and the first correspondence relationship.

6. The method for adjusting a detection position according to claim 5, wherein the acquiring a first correspondence relationship between ultrasonic images and detection positions of the detected object specifically comprises:
determining the detected object corresponding to the first ultrasonic image; and
acquiring, according to the detected object, the first correspondence relationship between ultrasonic images and detection positions of the detected object.

7. The method for adjusting a detection position according to claim 6, wherein the determining the detected object corresponding to the first ultrasonic image specifically comprises:
determining the detected object corresponding to the first ultrasonic image according to the first ultrasonic image and a second correspondence relationship between ultrasonic images and the detected object, wherein the second correspondence relationship is determined in advance according to at least two ultrasonic images corresponding to the detected object.

8. The method for adjusting a detection position according to claim 6, wherein determining the detected object corresponding to the first ultrasonic image specifically comprises:
matching the first ultrasonic image with pre-stored first three-dimensional models for respective objects to be detected to determine a first three-dimensional model that is successfully matched, and taking an object to be detected corresponding to the first three-dimensional model that is successfully matched as the detected object corresponding to the ultrasonic image.

9. The method for adjusting a detection position according to claim 1, wherein the determining indication information for indicating movement of the ultrasonic probe, according to the current detection position of the detected object and a target detection position of the detected object specifically comprises:
calculating information on a relative position between the current detection position and the target detection position of the detected object; and
taking the information on the relative position as the indication information.

10. The method for adjusting a detection position according to claim 1, wherein the adjusting the detection position of the ultrasonic probe according to the indication information specifically comprises:
sending the indication information to the ultrasonic probe, outputting the indication information by the ultrasonic probe, and adjusting, by a user, the detection position of the ultrasonic probe according to the indication information; or, outputting the indication information, and adjusting, by a user, the detection position of the ultrasonic probe according to the indication information.

11. The method for adjusting a detection position according to claim 10, further comprising performing the following step after saving the first ultrasonic image:
determining whether there is a next target detection position for the detected object; and
if yes, determining indication information for indicating the movement of the ultrasonic probe to the next target detection position according to the current detection position of the detected object; or
if not, outputting the saved ultrasonic image corresponding to each target detection position.

12. The method for adjusting a detection position according to claim 1, further comprising performing the following step after determining the indication information for indicating movement of the ultrasonic probe:
if the indication information is determined as indicating that the current detection position of the detected object is the same as the target detection position of the detected object, saving the first ultrasonic image.

13. The method for adjusting a detection position according to claim 1, wherein the indication information comprises: a direction for the ultrasonic probe to adjust and an angle for the ultrasonic probe to rotate.

14. An ultrasonic probe, comprising:
at least one processor; and
a memory communicatively coupled to the at least one processor;
wherein the memory stores instructions executable by the at least one processor, the instructions being executed by the at least one processor to enable the at least one processor to perform the following steps:
acquire an ultrasonic signal of a current detection position;
send the ultrasonic signal, and receive indication information for indicating movement of the ultrasonic probe; and
output the indication information.

15. The ultrasonic probe according to claim 14, further comprising an indicator light;
wherein the instructions when being executed by the at least one processor further enables the at least one processor to control the indicator light to be turned on or turned off according to the indication information.

16. A terminal, comprising:
at least one processor; and
a memory communicatively coupled to the at least one processor;
wherein the memory stores instructions executable by the at least one processor, the instructions being executed by the at least one processor to enable the at least one processor to perform the method for adjusting a detection position, comprising steps of:
acquiring a first ultrasonic image currently detected by an ultrasonic probe;
determining a current detection position of a detected object corresponding to the first ultrasonic image;
determining indication information for indicating movement of the ultrasonic probe, according to the current detection position of the detected object and a target detection position of the detected object; and
adjusting the detection position of the ultrasonic probe according to the indication information.

17. The terminal according to claim 16, wherein the determining a current detection position of a detected object corresponding to the first ultrasonic image specifically comprises:
determining a detected object corresponding to the first ultrasonic image; and
determining position information corresponding to an area of the detected object that matches the first ultrasonic image, and taking a position indicated by the position information as the current detection position of the detected object corresponding to the first ultrasonic image.

18. The terminal according to claim 16, wherein the determining a current detection position of the detected object corresponding to the first ultrasonic image specifically comprises:
acquiring a first correspondence relationship between ultrasonic images and detection positions of the detected object; and
determining the current detection position of the detected object corresponding to the first ultrasonic image, according to the first ultrasonic image and the first correspondence relationship.

19. The terminal according to claim 18, wherein the acquiring a first correspondence relationship between ultrasonic images and detection positions of the detected object specifically comprises:
determining the detected object corresponding to the first ultrasonic image; and
acquiring, according to the detected object, the first correspondence relationship between ultrasonic images and detection positions of the detected object.

20. The terminal according to claim 18, wherein the determining the detected object corresponding to the first ultrasonic image specifically comprises:
determining the detected object corresponding to the first ultrasonic image according to the first ultrasonic image and a second correspondence relationship between ultrasonic images and the detected object, wherein the second correspondence relationship is determined in advance according to at least two ultrasonic images corresponding to the detected object.
